(19) United States
(12) Patent Application Publication
(10) Pub. No.: US 2011/0091930 A1
(43) Pub. Date: Apr. 21, 2011

(54) WELL-BASED FLOW SYSTEM FOR CELL CULTURE

(75) Inventors: Joseph P. Vacanti, Winchester, MA (US); Howard I. Pryor, Washington, DC (US); Craig M. Neville, Melrose, MA (US); Ira Spool, Boston, MA (US)

(73) Assignee: The General Hospital Corporation, Boston, MA (US)

(21) Appl. No.: 12/866,970

(22) PCT Filed: Feb. 13, 2009
(86) PCT No.: PCT/US2009/000915

§ 371 Date: Dec. 13, 2010

Related U.S. Application Data
(60) Provisional application No. 61/065,685, filed on Feb. 14, 2008.
(60) Provisional application No. 61/130,077, filed on May 28, 2008.

(51) Int. Cl. C12Q 1/20; C12M 3/00; C12N 5/071

(52) U.S. Cl. 435/33; 435/289.1; 435/307.1; 435/401; 435/373

(57) ABSTRACT

A well-based flow system for cell culture is described which provides for flow of culture containing compounds for drug screening to be exposed to cells seeded on a membrane. The flow of medium may be planar or radial and means are provided for the removal of waste media through fluid outlets in fluid communication with the assay well plates through conduits. Methods of using the system for cell culture and drug toxicity screening are also provided including coculturing cells such as hepatocytes, stem cells, fibroblasts and smooth muscle cells and selectively exposing cells to test compounds.

### CROSS-REFERENCE TO RELATED APPLICATIONS

This application claims priority to U.S. provisional patent application Ser. Nos. 61/065,685, filed Feb. 14, 2008, and 61/130,077, filed May 28, 2008, the entire contents of which are incorporated herein by this reference.

### STATEMENT REGARDING FEDERALLY SPONSORED RESEARCH

Not Applicable.

### BACKGROUND OF THE INVENTION

This invention is in the general field of cell culture systems and, more specifically, to a method and system for providing a continuous, controlled medium flow to individual wells of a multi-well cell culture apparatus.

In cell culture, it is often desirable to maintain cells in vitro for an extended time, during which the cultured cells produce waste, toxify the medium, and use up nutrients from the medium. The exhaustion of the medium is accelerated when cultured cells proliferate and/or differentiate into highly metabolic cell types. Thus, a general concern in cell culture is providing a means to refresh the culture medium without disturbing the cells. Spinner flasks, rotary devices, profusion bioreactors, and fluid sheer chambers have all been used to enhance nutrient and metabolite diffusion to and from cultured cells. In addition, the cell culture generates signals and metabolic waste that accumulates proportionally between medium changes. This accumulation can lead to derangements in cellular function and such derangements can obscure observations that require a significant period of time to develop in culture.

Cell growth in vitro on a microporous membrane suspended in a nutrient rich culture medium has several advantages. Cell layers may be seeded and readily grown on microporous membranes. The cells are fed through the semi-permeable membrane as a concentration gradient of nutrients develops between the two sides of the semi-permeable membrane. This method of providing nutrients to cells, termed “basolateral feeding” more closely resembles the situation in vivo, where, for example, a plane of attachment of smooth muscle tissue to an underlying endothelial tissue is also the path of nutrient exchange. Cells are fed as nutrients pass from the nutrient medium through the membrane and to the cells at a rate controlled by the concentration gradient of nutrients from the medium to the cells.

To take advantage of the basolateral feeding method, cell culture inserts using a permeable membrane as culture substrate have been developed. The inserts fit into reservoirs or chambers of a culture plate, such that each membrane is immersed in the culture medium in a corresponding reservoir. Such devices generally provide access to the culture medium below the permeable membrane, advantageous for maintaining appropriate levels of nutrients and waste products in the culture medium. Through these openings, culture medium can be removed and replaced without disturbing the membrane and cultured cells associated therewith. Various multiwell plate systems that include permeable supports or membranes are known in the art and find use in many types of studies, including drug discovery.

In the field of drug discovery, the use of primary human cells to study the absorption, distribution, metabolism, elimination and toxicity of a drug candidate is highly desirable. This is due to the fact that use of whole animals is expensive, and results are not always predictive of responses in man. In vitro study of primary human cells is attractive due to the economics of the approach, and the fact that data from human cells is regarded as more relevant than animal data. Unfortunately, the culture of primary human cells is extremely difficult for most cell types, and there are few model systems that are capable of creating relevant models of in vivo tissues and organs.

Therefore, there is a great need for bioreactor-type devices that provide nutrient and metabolite transport in a manner akin to in vivo tissues and organs, while maintaining a high throughput parallel testing format. It can be appreciated that a demand exists for systems and methods for culturing cells under fluid profusion in high throughput format, to assay the abilities of cells to respond to chemical or physical cues and to facilitate the systematic assessment of cell growth and/or differentiation.

### BRIEF SUMMARY OF THE INVENTION

The present invention provides a cell culture system capable of providing nutrient and metabolite transport in a manner akin to in vivo tissues and organs, while maintaining a high throughput parallel testing format.

One aspect of the present invention provides a well-based flow system for cell culture including an input, a flow assembly in fluid communication with the input, the flow assembly configured to provide flowing medium to a cell culture insert, and an output in fluid communication with the flow chamber to drain medium from the flow chamber. The flow chamber can be configured to generate a radial flow medium flow path or a planar flow medium flow path.

In another aspect, the present invention provides a well plate for holding a plurality of cell culture inserts including a membrane. The well plate can include a plurality of inlets positioned on an outside perimeter of the well plate, a plurality of inlet conduits each connected to one of the plurality of inlets, a plurality of wells each connected to one of the plurality of inlet conduits and each including a flow assembly and a flow chamber, the flow assembly configured to provide flowing medium to the flow chamber, plurality of outlet conduits each connected to one of the plurality of wells, and a plurality of outlets on the outside perimeter of the well plate, each of the plurality of outlets connected to one of the plurality of outlet conduits. The plurality of wells can be configured to generate a radial flow medium flow path or a planar flow medium flow path in the flow chamber.

In another aspect, the invention is provides a method of culturing cells using a well-based flow system of the invention. Such a method includes steps of: (a) providing a well-based flow system having a cell culture insert, as described herein; (b) seeding cells to be cultured on a membrane of said cell culture insert; and (c) positioning the cell culture insert in the well-based flow system such that a flow of culture medium is directed to the flow side of the cell culture insert. Cells to be cultured are maintained on the membrane of the cell culture insert. The invention provides that the flow of culture medium directed to the flow side of the cell culture insert may be configured in a radial or, alternatively, a planar flow pattern.

In another aspect, the present invention provides a method of culturing primary cells in the presence of a secondary co-culture layer. Such a method includes steps of: (a) providing a well-based flow system having a cell culture insert, as described herein; (b) seeding a secondary co-culture cell layer on a membrane positioned at a flow side of the cell culture insert; (c) seeding primary cells to be cultured on the opposite side of the membrane of the cell culture insert, the secondary co-culture layer and primary cells to be cultured opposing each other across the membrane of the cell culture insert; and (d) positioning the cell culture insert in the well-based flow system such that a flow of culture medium is directed to the flow side of the cell culture insert. The primary cells to be cultured are maintained in the cell culture insert. Cell-cell interactions through contact and/or soluble factors are regulated by the physical characteristics of the provided membrane.

In certain embodiments, the flow of culture medium directed to the flow side of the cell culture insert is a radial flow of culture medium, which can be reversible (i.e. center-to edge or edge-to-center). Alternatively, the flow of culture medium directed to the flow side of the cell culture insert may be a planar flow of culture medium.

A wide variety of cell types may be cultured in a flow system according to the invention including, but not limited to, cell types best grown in the presence of a secondary co-culture, such as a cell feeder layer. For example, a flow system according to the invention may be used to: culture stem cells in the presence of a cell feeder layer of fibroblasts; culture hepatocytes in the presence of a secondary co-culture layer of endothelial cells or stellate cells; or muscle cells in the presence of a secondary co-culture layer of endothelial cells.

In yet another aspect, the invention is provides an in vitro method of assaying liver metabolism, particularly toxicity, of a chemical entity. Such a method includes steps of: (a) providing a well-based flow system having a cell culture insert, as described herein; (b) seeding endothelial cells on a membrane positioned at a flow side of the cell culture insert; (c) seeding hepatocytes on an opposite side of the membrane of the cell culture insert, the endothelial cells and hepatocytes opposing each other across the membrane of the cell culture insert; (d) positioning the cell culture insert in the well-based flow system such that a radial flow of culture medium is directed to the endothelial cells at the flow side of the cell culture insert; (e) including a chemical entity to be assayed for liver toxicity in the culture medium being directed by radial flow to the flow side of the cell culture insert; and (f) quantitating death of hepatocytes in the cell culture insert relative to a control cell culture insert prepared by steps (a)-(d) but not exposed to the chemical entity. Liver toxicity is thusly indicated by increased death of hepatocytes in the cell culture insert exposed to the chemical entity relative to the control cell culture insert.

In a further aspect, the present invention encompasses an in vitro method of modeling the physiological barrier provided by the vasculature (e.g. the internal elastic lamina and the blood-brain barrier) and, particularly, for assaying drug-induced vascular injury (DIVI) of a chemical entity. Such a method includes steps of: (a) providing a well-based flow system having a cell culture insert, as described herein; (b) seeding endothelial cells on a membrane positioned at a flow side of the cell culture insert; (c) seeding smooth muscle cells on an opposite side of the membrane of the cell culture insert, the endothelial cells and smooth muscle cells opposing each other across the membrane of the cell culture insert; (d) positioning the cell culture insert in the well-based flow system such that a planar flow of culture medium is directed to the endothelial cells at the flow side of the cell culture insert; (e) including a chemical entity to be assayed for DIVI in the culture medium being directed by planar flow to the flow side of the cell culture insert; and (f) evaluating DIVI in the smooth muscle cells and endothelial cells of the cell culture insert relative to a control cell culture insert prepared by steps (a)-(d) but not exposed to said chemical entity. DIVI is indicated by increased pathology in the smooth muscle cells and endothelial cells of the cell culture insert exposed to the chemical entity relative to the control cell culture insert. For example, detection of medium-born compounds in the chamber of the cell culture insert would be indicative of a breakdown of the barrier provided by the endothelial cells.

In yet another aspect, the invention provides kits comprising the well-based flow system and instructions for use. In certain embodiments, the instructions direct the use of the well-based flow systems of the invention in accordances with the various methods of use described herein above.

As can be appreciated, it is one object of the present invention to provide a well-based flow system which provides nutrient and metabolite transport in a manner akin to in vivo tissues and organs, while maintaining a high throughput parallel testing format. This invention provides the advantage over prior technologies in that embodiments of the invention utilize or are based on improved fluidic routing, as recently developed by the present inventors and described herein. Other objects, features and advantages of the present invention will become apparent after review of the specification, claims and drawings.

### BRIEF DESCRIPTION OF THE DRAWINGS

FIG. 1 is a perspective view of a multi-well cell culture plate including discrete fluidic networks;

FIG. 2 is another perspective view of the multi-well cell culture plate of FIG. 1;

FIG. 3 is a close-up perspective view of a corner of the multi-well cell culture plate of FIG. 1;

FIG. 4 is a perspective view of a radial flow well showing a medium flow path through the well;

FIG. 5 is a cutaway view of the well of FIG. 4;

FIG. 6 is a close-up perspective view of the well of FIG. 4 showing a medium flow path through the well;

FIG. 7 is a side view of the well of FIG. 4 showing a medium flow path through the well;

FIG. 8 is a bottom view of the well of FIG. 4 showing a medium flow path through the well;

FIG. 9 is a side view of a radial flow well showing a medium velocity through the medium flow path;

FIG. 10 is a bottom view of a radial flow well showing a medium velocity across a membrane of a culture insert;

FIG. 11 is a perspective view of a prototype of a multi-well cell culture plate including radial flow wells and planar flow wells;

FIG. 12 is a sectional view of a planar flow well showing a medium velocity through the medium flow path;

FIG. 13 is a bottom view of a planar flow well showing a medium velocity across a membrane of a culture insert;

FIG. 14 is a photograph of a prototype of a multi-well cell culture plate including radial flow wells and planar flow wells;

FIG. 15 is an image showing a flow of florescent microbeads in a radial flow well; and

FIG. 16 is an image showing a flow of florescent microbeads in a planar flow well.

### DETAILED DESCRIPTION OF VARIOUS EMBODIMENTS

Before the present invention is described, it is understood that this invention is not limited to the particular materials, methodology, protocols, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

The term “obtaining” as in “obtaining the well-based flow system” recited in the appended claims is intended to include purchasing, synthesizing or otherwise acquiring the well-based flow systems according to the invention.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, certain methods and materials are now described. All publications and patents specifically mentioned herein are incorporated by reference for all purposes including describing and disclosing the materials, chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The practice of the present invention will employ, unless otherwise indicated, conventional techniques of tissue culture, molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

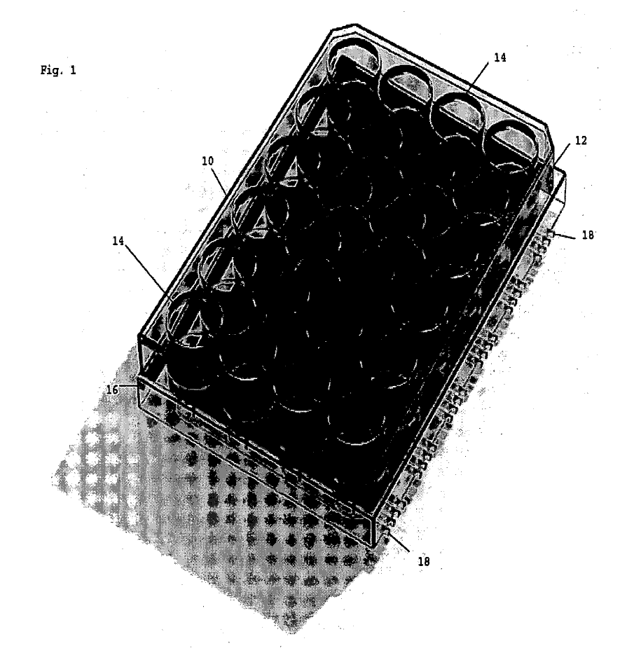

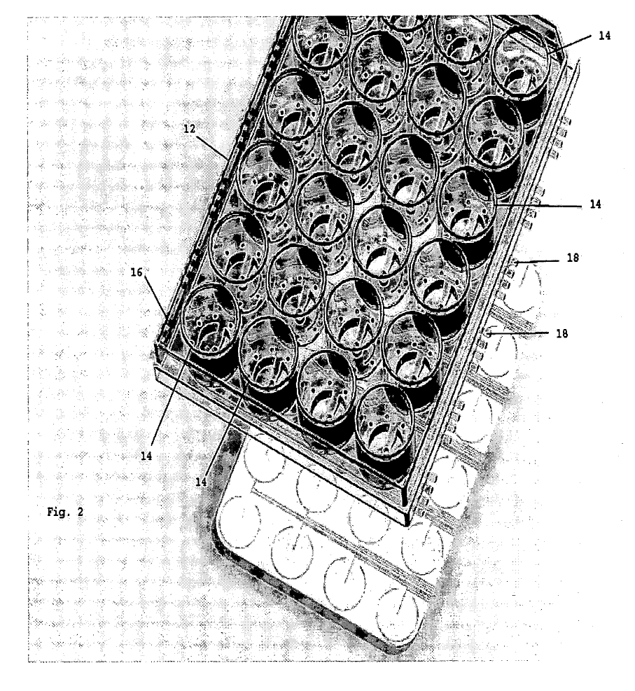

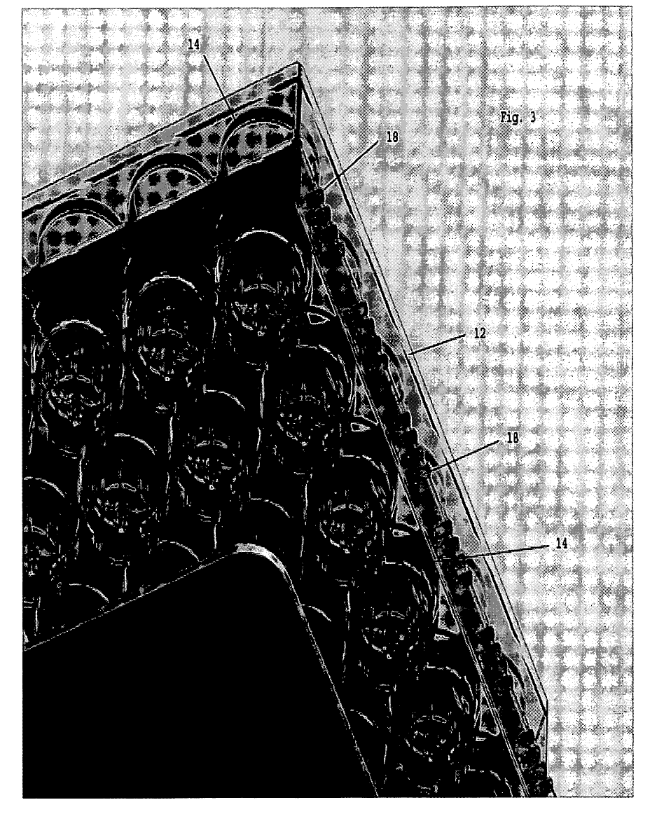

Reference is now made to FIGS. 1-3 which show a multi-well cell culture plate 10 including a flow system according to an embodiment of the present invention. Plate 10 includes a body 12, in which are formed a plurality of bioreactor wells 14, a plurality of supply lines 16, and a plurality outlet lines 18. As discussed below, wells 14 are configured to hold cell culture inserts that include a structure for seeding cells and other biological materials. Supply lines 16 supply medium to wells 14 and outlet lines 18 drain medium from wells 14. Plate 10 is shown with 24 bioreactor wells but can be scaled to include any number of wells, such as 1, 6, 8, or 96 wells. A single well version can be scaled up considerably for use in culturing large sheets of cells (e.g., 500 cm diameter). Plate 10 can comprise a single piece or multiple pieces joined together.

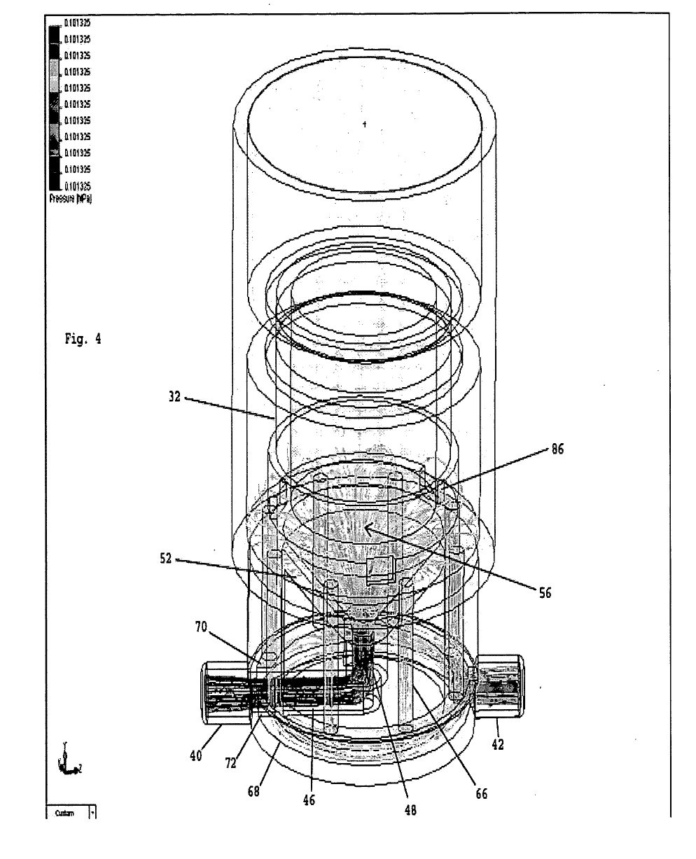

Referring now to FIGS. 4-6, a well 30 provides medium to a cell culture insert 32. Well 30 includes a base section 34 and an insert section 36. Base section 34 is cylindrical about a central axis 38. Base section 34 includes an inlet 40 for introducing medium to well 30 and an outlet 42 for draining medium from well 30. Inlet 40 and outlet 42 are tubular, share an axis 44, can have the same internal radius, are positioned at the bottom of base section 34, and extend from opposite sides of base section 34. An inlet conduit 46 and a discharge conduit 48 are formed in base section 34. Inlet conduit 44 is tubular and shares axis 44. Discharge conduit 48 is tubular and shares central axis 38, which is perpendicular to axis 44. Inlet conduit 46 extends from inlet 40 to discharge conduit 48. Inlet 40, inlet conduit 46, and discharge conduit 48 are in fluid communication. A rounded discharge mouth 50 flares from the end of discharge conduit 48. A flow chamber wall 52 extends from discharge mouth 50. Flow chamber wall 52 is frusto-conical and centered around central axis 38. Flow chamber wall 52 opens away from discharge mouth 50, with its widest point defined by an annular flow chamber edge 54. The configuration of flow chamber wall 52 can be changed to produce desired flow paths and flow velocity. Flow chamber wall 52 defines a portion of a flow chamber 56. An annular drain ledge 58 extends from flow chamber edge 54 to an annular well wall edge 60 adjacent a well wall 62. Drain ledge 58 defines a plane that is perpendicular to central axis 38. Well wall 62 is cylindrical about central axis 38 and defines an insert cavity 64. Well wall 62 extends to the top of well 30. A plurality of drain conduits 66 extend through drain ledge 58 to a drain channel 68 that is in fluid communication with outlet 42. Drain conduits 66 are tubular and each have an axis that is parallel to central axis 38. Drain conduits 66 are spaced equally around the drain ledge 58. In an embodiment, well 30 includes eight drain conduits. Drain channel 68 starts at a channel end 70 then wraps around the perimeter of base section 34 and terminates at a channel end 72. Drain channel is formed in the layer of base section 34 in which inlet conduit 46 is formed. Channel ends 70,72 are positioned on opposite sides of inlet conduit 46. Hence, well 32 provides a flow path for introducing and draining medium. Medium can flow into inlet 40, through inlet conduit, through discharge conduit 48, out discharge mouth 50, through flow chamber 56, through drain conduits 66, through drain channel 68, and through outlet 42.

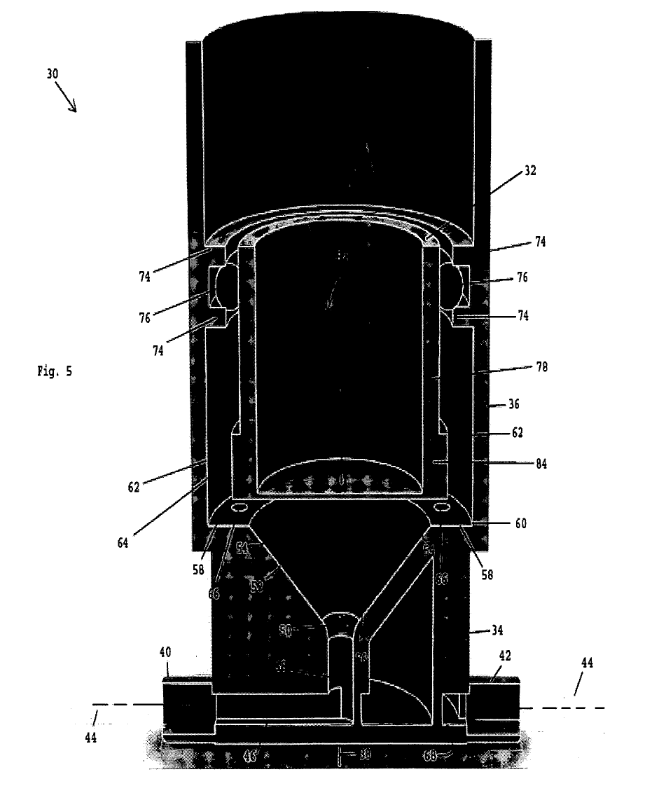

Well 30 includes a pair of annular sealing protrusions 74 that extend inwardly from well wall 62. A sealing member 76 is seated between sealing protrusions 74 and seals against cell culture insert 32 when cell culture insert 32 is positioned in insert cavity 64. Cell culture insert 32 includes a cylindrical insert wall 78 that is open at the top and closed at the bottom by a membrane 80. Insert wall 78 and membrane 80 define an insert chamber 82. A flange 84 extends from insert wall 78 adjacent its bottom. A plurality of feet 86 extend from the bottom of cell culture insert 32, as shown in FIG. 5. When cell culture insert 32 is positioned in well 30, feet 86 rest on drain ledge 58 to provide spacing between drain ledge 58 and the bottom of insert 32. The spacing allows for medium to flow into drain conduits 66. Alternatively, cell culture insert 32 can be a hanging insert or other type of insert that does not require feet in order to provide adequate spacing for fluid flow and drainage. Moreover, the drain ledge can include a plurality of spacers that the insert sits upon to provide adequate spacing for fluid flow and drainage.

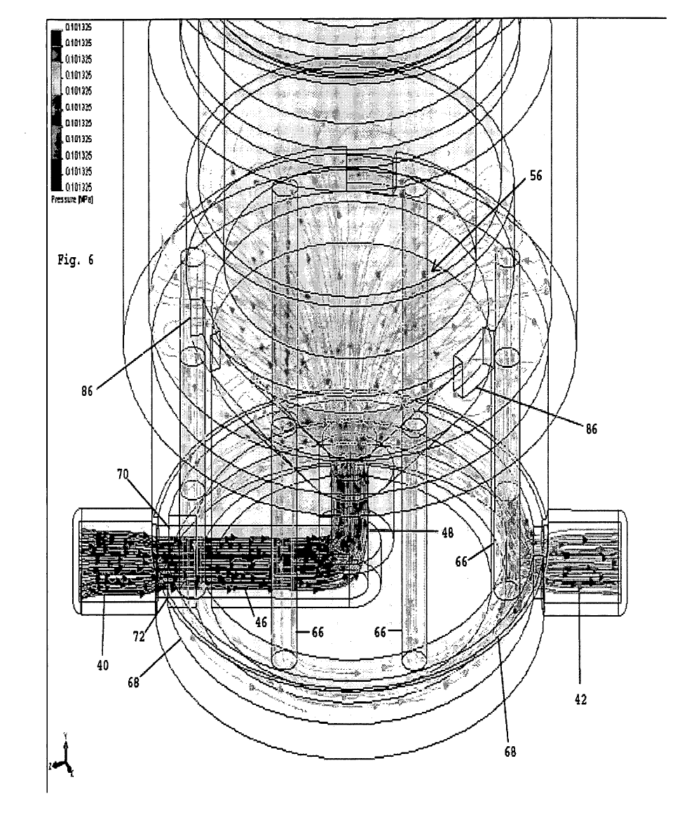

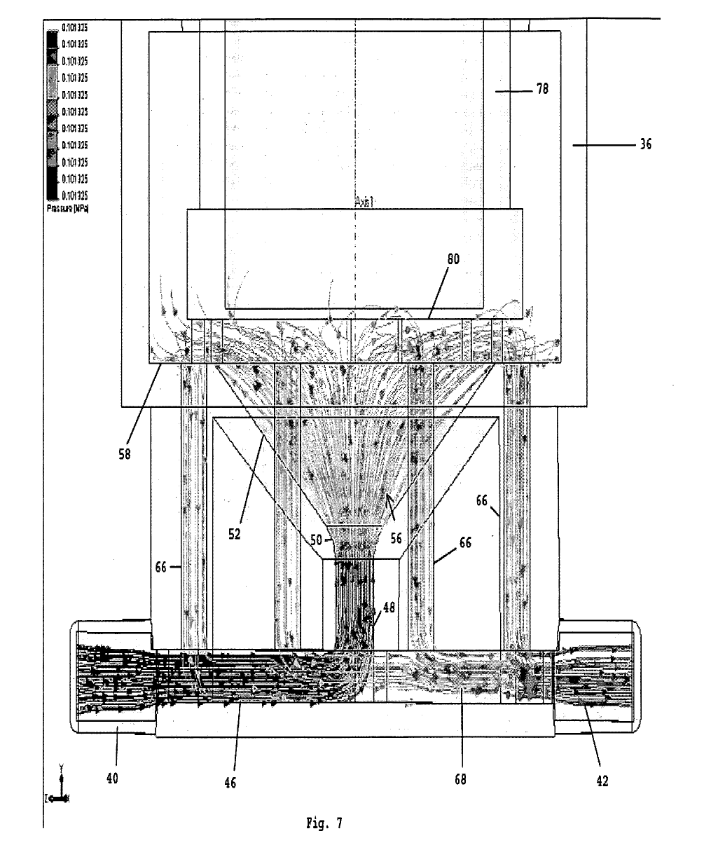

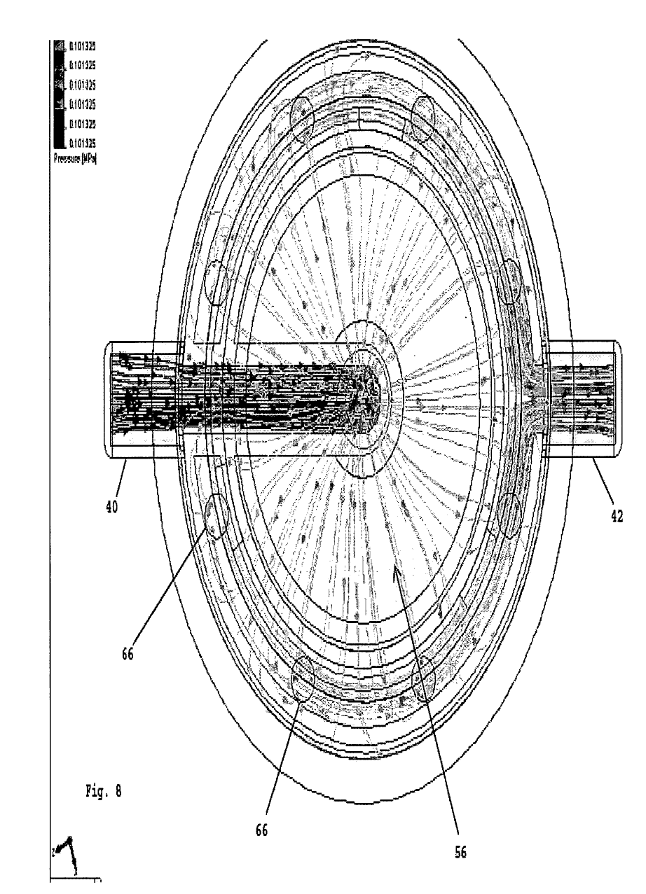

Referring to FIGS. 6-8, well 30 provides for radial flow of medium. Medium enters flow chamber 56 at discharge mouth 50, which is at the center and bottom of flow chamber 56. Medium then flows upward toward the bottom surface of membrane 80. Medium then fans out over the bottom surface of membrane 80 and flows towards the perimeter of well 30. Medium is then collected via drain conduits 66. Flow can be also directed in the reverse direction (e.g., edge-to-center or center-to-edge.

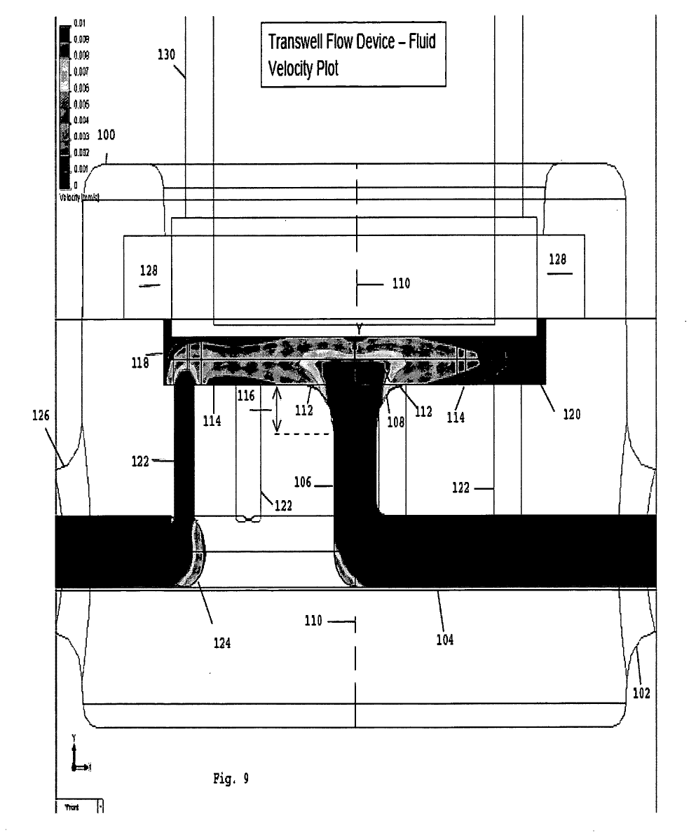

FIGS. 9-11 show a well 100 that provides for radial flow of medium according to another embodiment of the present invention. Well 100 includes an inlet 102, an inlet conduit 104, a discharge conduit 106, and a discharge mouth 108. Discharge conduit is symmetric about a central axis 110 that is located at the center of well 100. Discharge mouth 108 is rounded and flares from the end of discharge conduit 106 and terminates at an annular edge 112 of a flow chamber floor 114. Discharge mouth 108 has a height 116. Flow chamber floor 114 is defined generally by a plane that is perpendicular to axis 110. Flow chamber floor 114 extends from annular edge 112 to a flow chamber wall 118, which is generally cylindrical about central axis 110. Annular edge 112 defines the widest portion of discharge mouth 108, which is symmetrical about central axis 110. In this embodiment, the radius of annular edge 112 is substantially less than the radius of flow chamber wall 118. A flow chamber 120 is at least partially defined by discharge mouth 108, flow chamber floor 114 and flow chamber wall 118. A plurality of drain conduits 122 are spaced equally around the perimeter of flow chamber 120. Drain conduits 122 each have an axis that is parallel to central axis 110. Drain conduits 122 extend through flow chamber floor 114 to a drain channel 124 that is in fluid communication with an outlet 126. Well 100 includes a seating flange 128 positioned above flow chamber wall 118. Seating flange 128 extends towards central axis 110. When a cell culture insert 130 is positioned in well 100, seating flange 128 abuts cell culture insert 130 to seal flow chamber 120. The feet of cell culture insert 130 rest upon flow chamber floor 114.

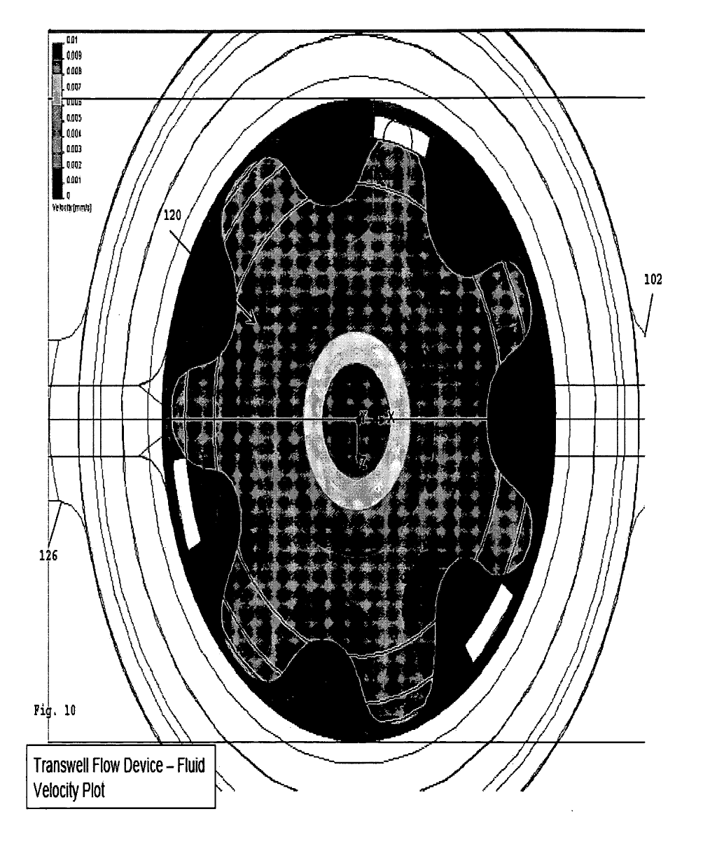

Hence, well 100 provides a radial flow path for introducing and draining medium that provides a profile of varying medium velocities across the membrane of the insert. The velocity of the medium determines the concentration of nutrients that are received by the cells. As illustrated in FIG. 10, the medium velocity at a given point adjacent the insert membrane generally depends on its distance from central axis 110 and/or drain conduits 122; therefore, well 100 produces generally annular zones of substantially common velocities. Accordingly, the cells positioned nearest central axis 100 will receive the highest concentration of nutrients from the medium and the cells positioned farthest from central axis 100 will receive the lowest concentration of nutrients. This scenario is reversed in an edge-to-center configuration. The edge-to-center configuration would result in a larger number of cells within the culture being exposed to the high nutrient content media (i.e., as there is more surface area at the perimeter). Likewise, the waste material concentrations will vary in diminishing or increasing gradient fashion. In alternate embodiments, height 116 of discharge mouth 108 and the radius of annular edge 112, which defines the maximum width of discharge mouth 108, can be varied to produce a desired flow path and/or profile of varying medium velocities. For example, widening discharge mouth 108 can increase the size of the highest velocity zone, which is indicated by the color red in FIG. 10.

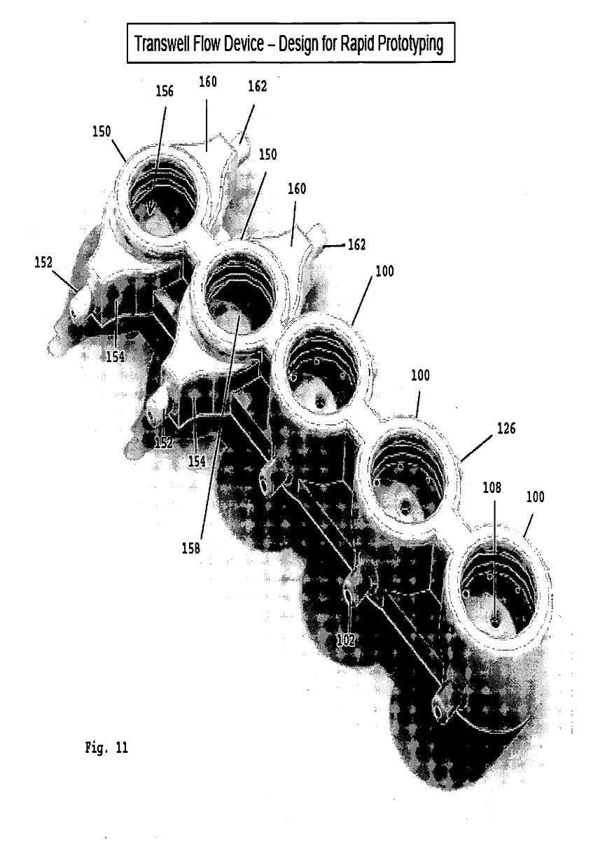

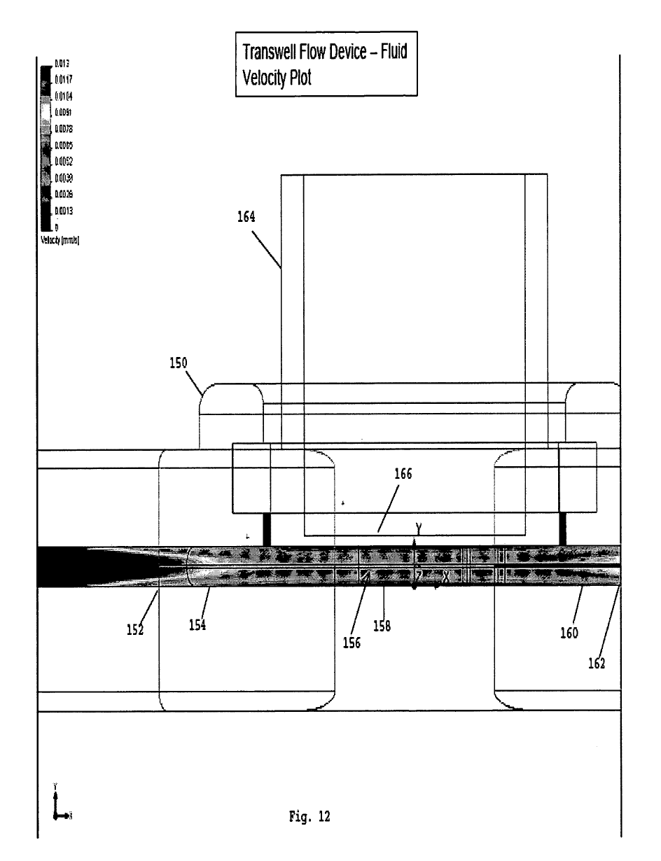

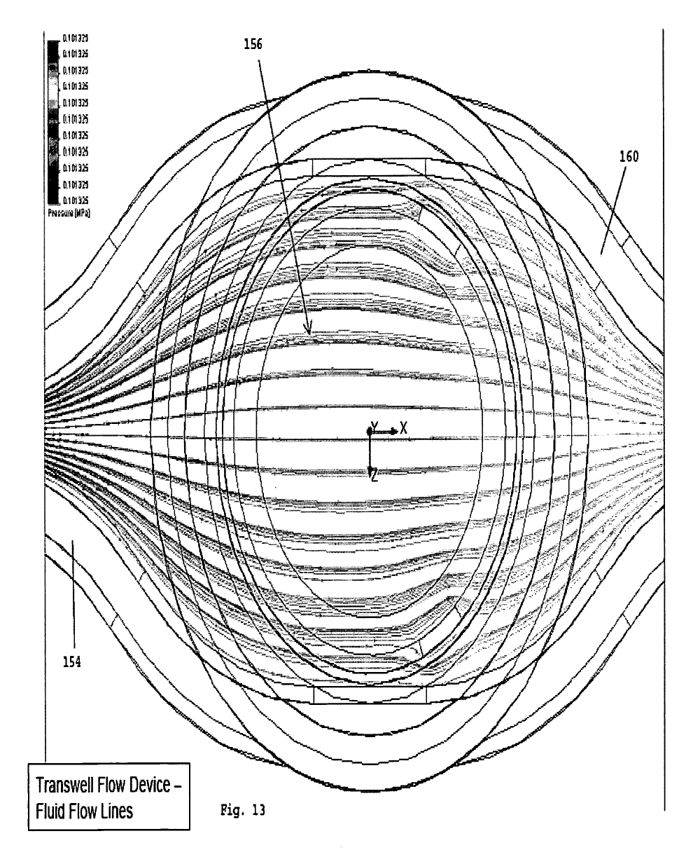

FIGS. 11-14 show a well 150 that provides for planar flow of medium according to another embodiment of the present invention. Well 150 includes an inlet 152 for providing medium to a planar flow discharge nozzle 154. Planar flow discharge nozzle 154 opens into a flow chamber 156 that is partially defined by a flow chamber floor 158. Flow chamber 156 empties into a planar flow receiver 160, which in turn is connected to an outlet 162. Planar flow receiver 160 is a mirror image version of planar flow discharge nozzle 154. When a cell culture insert 164 including a membrane 166 is positioned in well 150, discharge nozzle 154 causes the medium to flow past membrane 166 in a planar flow path. As shown in FIG. 12, the planar flow path results in a substantially consistent medium velocity within flow chamber 156. The consistent velocity through flow chamber 156 provides for consistent nutrient and waste material concentrations. The consistent velocity within flow chamber 156 can be varied for different studies by varying the velocity of the medium supplied to inlet 152, which can be useful for vascular studies. The planar flow path can be substantially laminar.

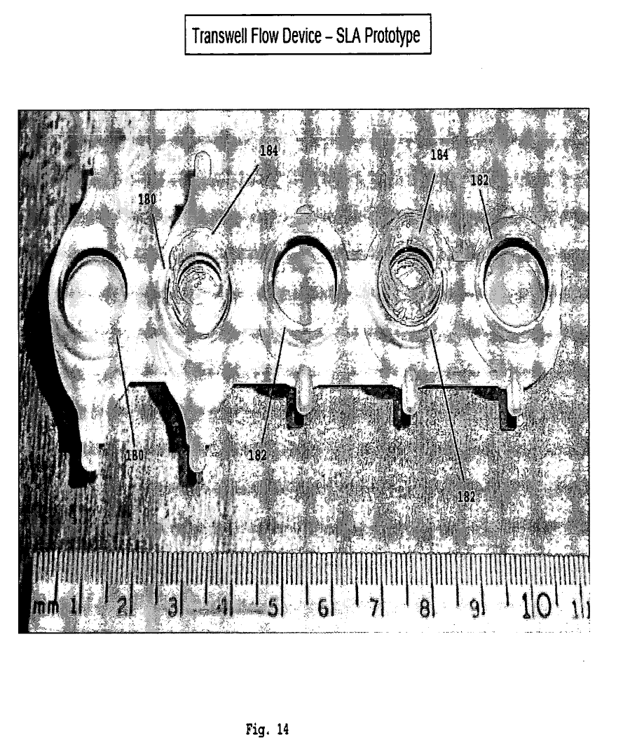

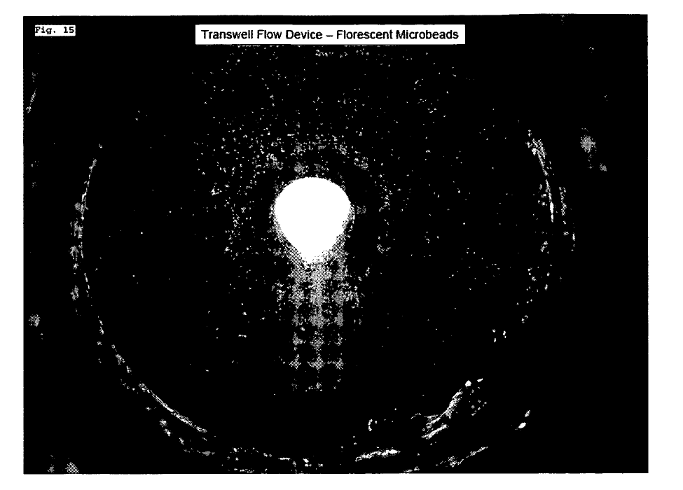

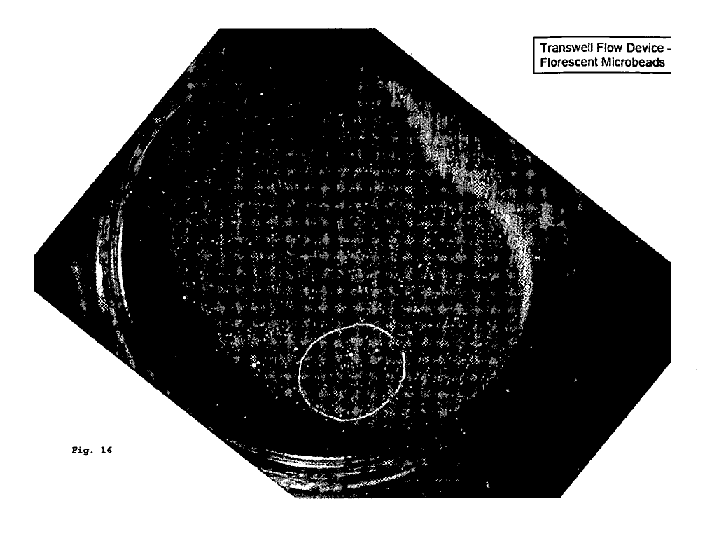

FIG. 14 shows a prototype including two planar-flow wells 180 and three radial-flow wells 182. Two cell culture inserts 184 are disposed in one of wells 180 and one of wells 182. FIG. 15 shows a fluorescent microbead evaluation of flow in radial-flow well 182. FIG. 16 shows a fluorescent microbead evaluation of flow in planar-flow well 180.

When arranged in a multi-well plate such as plate 10 of FIGS. 1-3, the inlet of a well is in fluid communication with one of supply lines 16 and outlet conduit of the well is in fluid communication with one of outlet lines 18. In this embodiment, each well has its own medium supply and outlet, which allows for separate experiments on one plate. Each wells' supply and outlet medium are segregated from the other wells on the plate, which allows for investigation of the contents and condition of each wells' medium both before and after passage through the culture system. Other embodiments can provide different supply and outlet line configurations. For example, a network of supply and outlet lines can be configured to connect a number of wells in serial, which allows the user to model in vivo tissue conditions where nutrient and waste material concentrations vary in diminishing or increasing gradient fashion as would be the case across a spatial dimension of an organ.

As can be appreciated, the well-based flow system described and claimed herein facilitates a method of cell culture in which nutrient and metabolite transport occurs in a manner akin to in vivo tissues and organs, while maintaining a high throughput parallel testing format. Such a cell culture method includes general steps of: (a) providing a well-based flow system having a cell culture insert, as described herein; (b) seeding cells to be cultured on a membrane of said cell culture insert; and (c) positioning the cell culture insert in the well-based flow system such that a flow of culture medium is directed to the flow side of the cell culture insert. Cells to be cultured are maintained on the membrane of the cell culture insert. The invention provides that the flow of culture medium directed to the flow side of the cell culture insert may be configured in a radial or, alternatively, a planar flow pattern. The flow system may be configured such that individual wells receive independent flows of culture medium or, alternatively, all or a pre-determined number of wells may be interconnected in series such that culture medium is passed serially, from well to well.

Exemplary uses of the above-described well-based flow system configured as a radial flow system will now be described. Using an inverted cell culture insert, endothelial cells may be seeded on the bottom of the insert in a static drop of culture. After a suitable seeding time (e.g., 1-6 hours), the insert is inverted to its original position and placed in the reactor. A second cellular population, for instance hepatocytes, is then seeded in the top chamber of the cell culture insert and the bioreactor is filled with medium. Medium flow through the reactor is initiated and the culture is maintained for the desired incubation time. In this example, endothelial cells are exposed to the flow condition of the bioreactor, a circumstance similar to physiologic conditions. Similarly, the hepatocytes are protected from the flow of the reactor while still experiencing the physiological benefits of a constantly renewed stream of nutrient-laden medium, which maintains the nutrient gradient across the membrane. In addition, the medium that diffuses through the membrane is conditioned by the normally excreted products of the endothelial cells, also a condition similar to expected physiological conditions. This conditioning is referred to as paracrine signaling and is a feature of in vivo tissues and organs that is not accurately reproduced in prior flow reactors.

Accordingly, the present invention also provides a method of culturing primary cells in the presence of a secondary co-culture cell layer. Such a method includes steps of: (a) providing a well-based flow system having a cell culture insert, as described herein; (b) seeding a secondary co-culture cell layer on a membrane positioned at a flow side of the cell culture insert; (c) seeding primary cells to be cultured on an opposite side of the membrane of the cell culture insert, the secondary co-culture cell layer and primary cells to be cultured opposing each other across the membrane of the cell culture insert; and (d) positioning the cell culture insert in the well-based flow system such that a flow of culture medium is directed to the flow side of the cell culture insert. The primary cells to be cultured are maintained on the membrane of the cell culture insert.

A reactor operating in radial flow configuration allows for several cell culture conditions that would be broadly useful in the biomedical and pharmaceutical industries. For example, the radial flow system configuration allows for the study of a three-zone population of hepatocytes. In a liver, sinusoid blood flows from the portal vein to a central collecting vein. The cells clustered around the portal vein receive the highest concentration of nutrients and oxygen, so called Zone I hepatocytes. The cells around the central vein receive blood with the least amount of cell and oxygen, so called Zone III hepatocytes. Zone II hepatocytes lie between these two regions. Each of the regions of hepatocytes has a unique profile of metabolic functions as a result of their usual supply of nutrients and oxygen. Direct hepatotoxicity can be demonstrated by death of hepatocytes in any of these three zones and the zone with the highest concentration of cellular demise can indicate the metabolic mechanism of hepatotoxicity. As can be appreciated from the foregoing, a radial flow system according to the present invention provides a platform which better models hepatocytes in the in vivo context, thereby offering a substantial advantage in high-throughput drug screening.

The invention, in one aspect, is directed to an in vitro method of assaying toxicity of a chemical entity. There are three general classes of toxicity. Acute toxicity is a toxic effect that occurs after less than about 24 hours of exposure to the drug. Subacute toxicity occurs later, after about 14 to 90 days of exposure to the drug. Chronic toxicity occurs after about 90 days (or longer) exposure to the drug. While methods of the invention can encompass these longer intervals of exposure, effects may be detected more rapidly, such that the incubation time for the test agent need not be extended. Accordingly, incubation times can range between about 1 hour to 24 hours, or can be extended as necessary for several days or even weeks. Detection of toxicity using standard methods known in the art can be employed with the methods of the invention. Such methods are described, for example, in U.S. application Ser. No. 11/183,115, filed on Jul. 15, 2005, the entire contents of which are incorporated herein by reference.

The undesired effects of toxicity caused by administration of a chemical entity can be screened in several ways. Culture systems of the invention can be used to determine the range of toxic dosimetry of a test agent. The effect of increasing concentrations of the chemical entity (i.e., dose) on tissues of interest can be monitored to detect toxicity. For example, different wells can be exposed to a slightly higher concentration of a chemical entity until a toxic concentration is demonstrated. A toxic effect, when observed, can be equated with a measurement of the concentration of a chemical entity/cells cm². By calculating the toxic concentration according to the distribution of cells in the culture system, one of skill in the art can extrapolate to the living system, to estimate toxic doses in subjects of various weights and stages in development. Indirect hepatoxicity, for example, can be demonstrated by treating the cells with a chemical entity and measuring their subsequent ability to metabolize a second chemical entity. Differences in metabolic rate indicate indirect hepatotoxicity where the function of the cell is impaired without causing death. Culture systems of the invention can also be linked in series, e.g., such that hepatocytes metabolize a chemical entity and the effluent from in that well flows to a serially connected well containing cells such as kidney, cardiac, embryologic or hematopoetic cells, which are assayed to evaluate indirect toxicity.

Using methods of the present invention, various doses of individual chemical entities and combinations of chemical entities with other pharmaceuticals will be screened to detect toxic effects, including but not limited to irregular metabolism, carcinogenicity and cell death. To detect irregular changes in metabolism, standard methods known in the art for assaying metabolite production, including but not limited to glucose metabolism and enzymatic assays, can be employed. The particular metabolic pathway assayed, or metabolite measured, can vary according to the tissue type selected.

In detecting carcinogenicity, cells can be screened for a transformed phenotype using methods well known in the art, for example, methods detecting changes in gene expression, protein levels, abnormal cell cycles resulting in proliferation and changes in expression of cell surface markers, including, but not limited to, antigenic determinants. Gene expression patterns can be determined, for example, by evaluating mRNA levels of genes of interest according to standard hybridization techniques, such as RT-PCR, in situ hybridization, and fluorescence in situ hybridization (FISH), Northern analysis or microchip-based analysis. Protein expression patterns can be determined by any methods known in the art, for example, by quantitative Western blot, immunohistochemistry, immunofluorescence, and enzyme-linked immunosorbent assay (ELISA), amino acid sequence analysis, and/or protein concentration assays. For details, see Sambrook, Fritsch and Maniatis, Molecular Cloning, A Laboratory Manual, 2nd Ed., Cold Spring Harbor Laboratory Press, 1989. Cell counting and/or separation techniques, such as FACS analysis, can be employed to measure proliferation or detect aberrant cell surface marker expression.

Standard methods well known in the art can also be used to detect cell death, including but not limited to, tunnel assays. Traditional approaches of in vitro toxicology to toxicological screening has been to measure comparatively late events in the process of cell death, such as lactate dehydrogenase release or differential counting of viable and dead cells using vital dyes, such as trypan blue, 4,6-diaminophenylindole (DAPI), propidium iodide, and LIVE/DEAD™ stain available from Molecular Probes. Prediction of lethality in vivo is one proposed application of this type of in vitro screen, although cell death is not a common mechanism by which the animal's death is induced following acute exposure to a toxic agent. In contrast, caspase activation is at the center the common features of chronic toxicity, cell death, hyperproliferation and inflammatory reactions. Caspase activity can be measured relatively quickly after a toxic insult (30 min to 4 hr) by fluorescence spectroscopy, thus lending itself to high-throughput screening techniques. Other markers and assays commonly used to monitor apoptosis or necrosis of cells can include, but are not limited to, the presence of phosphatidylserine on the outer leaflet of the plasma membrane of affected cells, annexin V staining, and terminal deoxynucleotidyltransferase nick-end labeling assay (TUNEL).

Using methods of the invention, various doses of individual chemical entities and combinations of test agents will be screened in panels comprised of cells having diverse genetic backgrounds to determine the pharmacogenetic toxicity profile of the test agents. For example, multiple doses of, or combinations with, test agents will be screened for toxic effects specific to one or more genetic backgrounds. Toxic effects to be screened for genetic variance include, but are not limited to, irregular metabolism, carcinogenicity and cell death.

Culture systems of the present invention can be modified in parallel to generate a comprehensive array of the currently known genetic polymorphisms of different metabolic enzymes. A salient example is the CYP450 monooxygenase system, wherein the population comprises multiple isoforms and polymorphisms that impinge on and complicate predictive models of drug metabolism, drug clearance, and toxicity. For example, in the metabolism of thiopurines, such as thioguanine, the rate-limiting enzyme is a methyltransferase that has different polymorphic forms. Polymorphism in the methyltransferases is known to affect metabolism of the thiopurines. Where the polymorphism gives rise to slower metabolism of the thiopurine, clinical benefit is decreased and where the polymorphism gives rise to an increased rate of metabolism, toxicity can result. Thus, methods of the invention can be used to determine the metabolic profile of various test agents in the presence of various polymorphic forms of an enzyme, such as methyltransferase.

In testing for differential toxicity due to polymorphic variation, or other genetic defects, genetically engineered cells comprising gene knockouts or knock-ins of specific enzymes known to affect drug metabolism and toxicity can be used in the systems of the invention. Cells can be modified using techniques that are known to the skilled artisan, such as RNA interference (RNAi), antisense technology, ribozymes, site-directed mutagenesis, among others.

In a specific embodiment for assaying liver toxicity, such a method includes steps of: (a) providing a well-based flow system having a cell culture insert, as described herein; (b) seeding endothelial cells on a membrane positioned at a flow side of the cell culture insert; (c) seeding hepatocytes on an opposite side of the membrane of the cell culture insert, the endothelial cells and hepatocytes opposing each other across the membrane of the cell culture insert; (d) positioning the cell culture insert in the well-based flow system such that a radial flow of culture medium is directed to the endothelial cells at the flow side of the cell culture insert; (e) including a chemical entity to be assayed for liver toxicity in the culture medium being directed by radial flow to the flow side of the cell culture insert; and (f) quantitating death of hepatocytes on the inside of the cell culture insert relative to a control cell culture insert prepared by steps (a)-(d) but not exposed to the chemical entity. Liver toxicity is thusly indicated, for example, by increased death of hepatocytes in the cell culture insert exposed to the chemical entity relative to the control cell culture insert.

In another specific embodiment, culture systems of the invention can be used to conduct methods of assaying for kidney toxicity. The kidney is a complex organ with an intricate vascular supply and at least 15 different cell types, which performs the critical functions of filtration, reabsorption and excretion. The basic functional unit of the kidney, the nephron, is composed of a vascular filter, the glomerulus, and a resorptive unit, the tubule. Filtration is dependent on flow and specialized glomerular endothelial cells. The majority (50-65%) of reabsorption is performed by the proximal tubule cells using active sodium transport through the energy-dependent Na⁺-K⁺-ATPase located on the basolateral membrane. Only 5-10% of the approximately one million nephrons in each human kidney is required to sustain normal excretory function. Toxicity in the kidney can occur, for example, as a result of allergic or hypersensitive immune responses to a chemical entity. The appearance of excess protein, such as albumin and creatinine, in the urine is indicative of toxicity. Thus, in assays for kidney toxicity conducted using the culture systems of the present invention, assays to detect toxicity of a particular chemical entity preferably comprise measurement of proteins including, but not limited to albumin. Such a method includes steps of: (a) providing a well-based flow system having a cell culture insert, as described herein; (b) seeding endothelial cells on a membrane positioned at a flow side of the cell culture insert; (c) seeding kidney cells on an opposite side of the membrane of the cell culture insert, the endothelial cells and kidney cells opposing each other across the membrane of the cell culture insert; (d) positioning the cell culture insert in the well-based flow system such that a radial flow of culture medium is directed to the endothelial cells at the flow side of the cell culture insert; (e) including a chemical entity to be assayed for kidney cell toxicity in the culture medium being directed by radial flow to the flow side of the cell culture insert; and (f) quantitating death of kidney cells on the inside of the cell culture insert relative to a control cell culture insert prepared by steps (a)-(d) but not exposed to the chemical entity. Kidney toxicity is thusly indicated, for example, by increased death of kidney cells in the cell culture insert exposed to the chemical entity relative to the control cell culture insert, or by abnormal production of albumin by kidney cells in the cell culture insert exposed to the chemical entity relative to the control cell culture insert.

The invention, in another aspect, is directed to an in vitro method of assaying metabolism of a chemical entity. For instance, the inflow could contain a substance that is metabolized exclusively by the liver and the outflow contains a product that is toxic to another cell type in a well connected in series to the liver well. Assays for monitoring enzyme metabolism, including cytochrome P450 enzyme function, can be performed according to methods well known in the art. Several of these assays are described below.

The cytochrome P450 superfamily of enzymes, which are primarily liver enzymes, catalyzes a wide variety of oxidative and reductive reactions and has activity towards a chemically diverse group of substrates. Cytochrome P450 enzymes are heme-containing membrane proteins localized in the smooth endoplasmic reticulum of numerous tissues. These hemoproteins are in close association with a second membrane protein, NADPH-cytochrome P450 reductase. Oxidative biotransformations catalyzed by cytochrome P450 monooxygenases include aromatic and side chain hydroxylation, N-, O-, and S-dealkylation, N-oxidation, sulfoxidation, N-hydroxylation, deamination, dehalogenation, and desulfuration. Cytochrome P450 enzymes, generally under conditions of low oxygen tension, also catalyze a number of reductive reactions. The only common structural feature of the diverse group of drugs oxidized by cytochrome P450 enzymes is their high lipid solubility.

A plurality of cytochrome P450 gene families has been identified in humans, and a number of distinct cytochrome P450 enzymes often exist within a single cell. The cytochrome P450 multigene family is classified by sequence similarity of the individual proteins. A given cytochrome P450 family is further divided into subfamilies, such that protein sequences within the same subfamily are >55% identical. The cytochrome P450 1, 2, 3, and 4 families (CYP1, CYP2, CYP3, CYP4) encode the enzymes involved in the majority of all drug biotransformations, while the gene products of the remaining cytochrome P450 families are important in the metabolism of endogenous compounds, such as steroids and fatty acids. The relevant CYP enzymes that are expressed in humans include, but are not limited to, CYP1A1, CYP1A2, CYP2A3, CYP2B6, CYP2B7, CYP2B8, CYP2C8, CYP2C9, CYP2C10, CYP2D6, CYP2D7, CYP2D8, CYP2E1, CYP2F1, CYP3A3, CYP3A4, CYP3A5, and CYP4B1. As a result of the relatively low substrate specificity among the cytochrome P450 proteins, two or more individual enzymes often can catalyze a given biotransformation reaction. CYP3A4 is involved in the biotransformation of a majority of all drugs and is expressed at significant level in the liver.

Cytochrome P450 is a hemoprotein that when reduced and complexed with carbon monoxide, a characteristic absorption spectrum results. The reduced carbon monoxide spectrum of cytochrome P450 absorbs maximally at around 450 nm and the extinction coefficient for the wavelength couple 450-490 nm has been accurately determined to be 91 mM.⁻¹ cm.⁻¹, thus allowing quantitative determination of this hemoprotein. If high turbidity is present in the sample containing cytochrome P450, spectrophotometric determination of the hemoprotein can be carried out in a split beam instrument, i.e. one containing both a sample and reference compartment to offset turbidity. Solid sodium dithionite, for example, is used as a reducing agent, and the samples can be gassed with carbon monoxide shortly after dithionite addition (the reduced ferrous form of CYP450 is relatively unstable). Excessively high gas flow rates can result in frothing and protein denaturation. If a prominent peak is observed at 420 nm after gassing with carbon monoxide, this is indicative of the presence of inactive cytochrome P420, and is to be avoided.

The tissue content of cytochrome b₅ can also be analysed using the same sample. If both cytochrome P450 and cytochrome b₅ concentration are required from the same sample, the cytochrome b₅ is advantageously determined first as in the method given below. This is achieved by determining the difference absorbance spectrum of NADH-reduced versus oxidized cytochrome b₅. The reduced, ferrous form of cytochrome b₅ has an absorbance maximum at 424 nm in difference spectrum and the extinction coefficient for the wavelength couple 424-490 nm is 112 mM⁻¹ cm⁻¹. NADH can be used as the reductant because of the presence of the flavoprotein enzyme NADH-cytochrome b₅ reductase in tissue preparations, an enzyme that relatively specifically and quantitatively reduces cytochrome b₅.

Many agents can bind to cytochrome P450, resulting in characteristic perturbations of the absorbance of the heme iron. The absorbance changes can be utilized to quantitatively describe drug binding to the hemoprotein, resulting in the determination of the apparent spectral dissociation constant (K_{S}) and maximum spectral change elicited by the drug (.DELTA.A.A.ₘₐₓ). These two parameters are formally similar to the Kₘ and Vₘₐₓ values described by Michaelis-Menten kinetics for enzyme-catalysed reactions. In the broadest sense, K_{S} is a measure of drug affinity for cytochrome P450 and ΔAₘₐₓ is the maximum spectral change. These two spectral parameters are therefore of use in comparing the interactions of test agents with various forms of cytochrome P450 or in comparing the interactions of different test agents, or combinations thereof, with the same form of cytochrome P450.

NADPH-cytochrome c (P450) reductase is a flavoprotein enzyme localized in the microsomal fraction of the liver that transfers the necessary reducing equivalents from NADPH to cytochrome P450 during certain drug metabolism reactions. As the reduction of cytochrome P450 is relatively difficult to assay directly, a simplified determination of enzyme activity is widely used, utilizing exogenous cytochrome c (oxidized, ferric form) as an artificial election acceptor. Accordingly, the reduction of cytochrome c by NADPH-cytochrome c (P450) reductase mirrors the reduction of cytochrome P450. The principle of the method is that oxidized (ferric) cytochrome c has a characteristic absorption spectrum, as does the reduced (ferrous) form. However, the reduced form has a characteristic absorption band at 550 nm, a band that is absent in the oxidized form. Therefore, the enzyme activity can be conveniently assayed by measuring the increase in absorbance at 550 nm as a function of time.

Many drugs are hydroxylated in the liver by the cytochrome P450-dependent, mixed-function oxidase system, and the 4-hydroxylation of aniline is a convenient, reproducible assessment of this reaction as: The 4-aminophenol metabolite produced is chemically converted to a phenolindophenol complex with an absorption maximum at 630 nm and is based on the method of Schenkman et al. Addition of aniline HCl solution initiates the enzyme reaction. The reaction is terminated with ice-cold 20% trichloroacetic acid and centrifuged to yield a clear solution (5 min in a bench centrifuge at maximum speed is usually sufficient). The supernatant can then be added to a 1% phenol solution in a separate test tube in the presence of sodium carbonate. After a 30-minute incubation, the absorbance is read at 630 nm.

N-demethylation of drugs is a common metabolic pathway and usually proceeds by initial hydroxylation at the α-carbon atom and subsequent breakdown of the carbinolamine intermediate liberating formaldehyde. Therefore, if the formaldehyde produced could be measured, this would then yield an appropriate assay for the N-demethylase activity. Formaldehyde may be trapped in solution as the semicarbazone and measured by the colorimetric procedure of Nash (1953), based on Hantzsch reaction. A solution including semicarbazide, MgCl.sub.2, and aminopyrine can be added to microsomes or post-mitochondrial supernatant, and the reaction occurs over 30 minutes. The reaction is terminated by addition of zinc sulfate on ice. A saturated barium hydroxide solution is added to the mix, and centrifuged to a clear supernatant. The Nash reagent is then added to the supernatant and incubated at 60° C. for 30 minutes. After cooling the tubes, the absorbance is read at 415 nm.

In a similar manner to N-demethylation, many drugs can undergo O-demethylation reactions, catalyzed by the microsomal, cytochrome P450-dependent, mixed-function oxidase system. A useful substrate to monitor O-demethylation reactions is 4-nitroanisole, which is converted to 4-nitrophenol as The 4-nitrophenol thus produced, forms an intense yellow color at pH 10, with an absorbance maximum at 400 nm. Hence the activity of the enzyme system can be followed spectrophotometrically.

A number of nonspecific esterases and amidases have been identified in the endoplasmic reticulum of liver, intestine, and other tissues. Such enzymes include acetylcholinesterase, pseudocholinesterase, other esterases, epoxide hydrolase, but are not limited to these examples. The alcohol and amino groups exposed following hydrolysis of esters and amides are suitable substrates for conjugation reactions. Epoxide hydrolase is found in the endoplasmic reticulum of essentially all tissues and is in close proximity to the cytochrome P450 enzymes. Epoxide hydrolase generally is considered a detoxification enzyme; hydrolyzing highly reactive arene oxides generated from cytochrome P450 oxidation reactions to inactive, water-soluble transdihydrodiol metabolites. Proteases and peptidase enzymes are widely distributed in many tissues and are involved in the biotransformation of polypeptide drugs.

The glucuronosyl transferase family of enzymes is important in phase II drug conjugation reactions. Uridine diphosphate glucuronosyltransferases (UDP-glucuronosyltransferases) catalyze the transfer of an activated glucuronic acid molecule to aromatic and aliphatic alcohols, carboxylic acids, amines, and free sulfhydryl groups of both exogenous and endogenous compounds to for O-, N-, and S-glucuronide conjugates. The UDP-glucuronosyltransferases are microsomal enzymes. Their location in the microsomal membrane facilitates direct access to the metabolites formed in phase I reactions. In addition to high expression levels in the liver, UDP-glucuronosyltransferases are also found in the kidney, intestine, brain, and skin.

A useful compound to assess glucuronosyl transferase activity is 2-aminophenol, because this phenol readily forms as O-linked glucuronide conjugate in the presence of UDP-glucuronic acid. The assay for glucuronidation of 2-aminophenol is based on the colorimetric diazotisation method for free primary amino groups. The principle of the analytical method is based on the observation that when an aqueous solution of sodium nitrite is added to a cold, acidified solution of an aromatic amine, a diazonium salt is formed. Excess nitrite is removed by the addition of ammonium sulfamate and the diazonium salt is finally reacted with a complex aromatic amine(N-naphthylethylene diamine), to produce a brightly coloured azo compound that can be analysed spectrophotometrically. This method, therefore, detects the amino group of the 2-aminophenyl glucuronide. The method is relatively specific because excess substrate (2-aminophenol) is destroyed under the assay conditions (at pH 2.7) and therefore does not take part in the diazotisation reaction.

As the glucuronosyl transferases usually exhibit enzyme latency in the microsomal membrane, the assay is carried out in the presence of a detergent (usually Triton X-100) to offset the latency. Ascorbic acid is included as an anti-oxidant. The substrate 2-aminophenol can be added to test samples comprising, for example, either microsomal or post-mitochondrial fraction, at 37° C. in a shaking water bath, and the reaction allowed to proceed for 30 minutes. The reaction is terminated by addition of ice-cold 20% trichloroacetic acid in phosphate buffer, pH 2.7, allowed to stand on ice for 5 minutes and clarified by centrifugation. Fresh 0.1% sodium nitrite is added, followed by 0.5% ammonium sulfamate, and 0.1% N-naphthylethylene diamine, incubated at room temperature in the dark for 60 minutes. The absorbance is read at 540 nm against the substrate blank.

Sulfation also is an important conjugation reaction for hydroxyl groups. Cytosolic sulfotransferases catalyze the transfer of inorganic sulfur from the activated 3′-phosphoadenosine-5′-phosphosulfate donor molecule to the hydroxyl group on phenols and aliphatic alcohols. Examples of sulfotransferases include, but are not limited to, phenol sulfotransferase, alcohol sulfotransferase, sterid sulfotransferase, and arylamine sulfotransferase.

UDP-glycosyltransferases transfer glucose moieties in a similar fashion that glucuronosyltransferases conjugate glucuronic acid to pharmacologic agents. Ribose and deoxyribose sugar moieties can also be added, mediated by enzymes such as purine phosphoribosyltransferase, among others.

A family of N-acetyltransferases is responsible for the acetylation of amines, hydrazines, and sulfonamides. In contrast to most drug conjugates, acetylated metabolites are often less soluble in water than the parent drug, a property that prolongs their elimination from the body. Conjugation of electrophilic metabolites with the tripeptide glutathione represents a major detoxification pathway for drugs and carcinogens.

The glutathione-S-transferases are a family of isoenzymes that catalyse the conjugation of the endogenous tripeptide glutathione (gamma-glutamylcysteinylglycine) with a large number of structurally diverse, electrophilic drugs or their metabolites. The glutathione S-transferase enzymes are expressed in virtually all tissues. Glutathione conjugates are cleaved to cysteine derivatives and subsequently are acetylated by a series of enzymes located primarily in the kidney to give N-acetylcysteine conjugates collectively referred to as mercapturic acids. The glutathione-S-transferases consist of two subunits each of which is inducible by many drugs, and although some exceptions are known, their prime function is in the detoxification of biologically reactive electrophiles.

A convenient spectrophotometric method has been developed for the analysis of glutathione-S-transferase activity based on the enzyme-catalyzed condensation of glutathione with the model substrate 2,4-dinitro-1-chlorobenzene. The product formed (2,4-dinitrophenyl-glutathione) absorbs light at 340 nm and the extinction coefficient of this product is known to be 9.6 mM.sup.-1 cm.sup.-1, thus facilitating the analysis of enzyme activity based on product formation. It known in the art that the glutathione-S-transferase isoenzymes have similar but overlapping substrate specificities for the electrophilic substrate to be conjugated. Therefore one substrate that is readily reactive with a particular isoenzyme may not be substrate for another isoenzyme. Dinitrochlorobenzene is a good substrate for most of the glutathione-S-transferase isoenzymes, when results are interpreted with the knowledge that observed activity can represent a composite result of the activity of each isoenzyme present in the tissue preparation. One skilled in the art can readily interpret the data to consider the results as a composite rather than an individual measure of metabolic activity.

A glutathione solution can be prepared in the presence of dinitrochlorobenzene and potassium phosphate buffer, pH 6.5. Since the reaction is measured as a function of time, the reaction is directly assayed in cuvettes placed in the spectrophotometer. The reaction is initiated by adding a post-mitochondrial or microsomal fraction from liver, mixed thoroughly, and the increase in absorbance at 340 nm over a 5 minute period should be measured as quickly as possible.

Methylation and conjugation with the amino acids glycine, glutamine, and taurine are less common reactions for drugs but represent important reactions for endogenous compounds. Methyltransferases include, but are not limited to, phenylethanolamine N-methyltransferase, non-specific N-methyltransferase, imidazole N-methyltransferase, catechol-O-methyltransferase, hydroxyindole-O-methyltransferase, and S-methyltransferase.

Other enzymes that are involved in drug metabolism, and that can be assayed in accordance with methods of the invention to determine the metabolic profile of a test agent, include, but are not limited to, alcohol dehydrogenase, aldehyde dehydrogenase, xanthine oxidase, amine oxidases such as monoamine oxidases, diamine oxidases, flavoprotein N-oxidases, and hydroxylases, aromatases, cysteine conjugate (β-lyase, α-galactosidase, (β-galactosidase, α-glucosidase, β-glucosidase, α-glucuronidase, β-glucuronidase, α-amylase, and alkylhydrazine oxidase.

Levels of metabolites, if known, can be detected using methods well known in the art as a reflection of metabolic activity, such as liquid chromatography. Liquid chromatography coupled with tandem mass spectrometric detection (LC/MS/MS) can be used as an analytical method to monitor early absorption, distribution, metabolism and elimination testing. This method provides excellent sensitivity, specificity and high sample throughput. The quantitative selectivity afforded by reaction monitoring on a triple quadrupole instrument precludes the need for high chromatographic resolution or extensive sample clean up. Using automated sample-processing techniques, such as on-line column switching, combined with high-sample-density microtiter plates, can further maximize analytical throughput. Modern LC/MS/MS also offers limits of detection extending down to the sub-nanogram per ml range using only minimal quantities of biological matrix.

LC/MS/MS enables rapid and sensitive quantitation of new drug candidates, as well as providing important structural information on metabolites. A full scan LC/MS analysis can initially suggest possible oxidative and/or conjugative metabolic transformations on the basis of the ionic species observed. In the MS/MS mode, the instrument can be tuned to a selected precursor ion of interest, which is then further fragmented to form productions that uniquely identify the metabolic (production scan).

Selectivity can be further enhanced by the quadrupole ion trap, a device that “traps” ions in a space bounded by a series of electrodes. The unique feature of the ion trap is that an MS/MS experiment (or, in fact, multi-step MS experiments) can be performed sequentially in time within a single mass analyzer, yielding a wealth of structural information. Hybrid quadrupole-time-of-flight (Q-TOF) LC/MS/MS systems can also be used for the characterization of metabolite profiles. The configuration of Q-TOF results in high sensitivity in mass resolution and mass accuracy in a variety of scan modes.

Liquid chromatography coupled with nuclear magnetic resonance spectroscopy (LC-NMR) provides a way of confirming absolute molecular configurations. A linear ion-trap mass spectrometer possesses significantly enhanced production-scanning capabilities, while retaining all of the scan functions of a triple quadrupole MS. The ultra-high resolution and sensitivity of Fourier transform ion-cyclotron resonance MS (FI-ICRMS) can be useful for the analysis and characterization of biological mixtures. Data processing and interpretation software packages also enable efficient identification and quantification of metabolites.

A widely used method to study in vitro drug metabolism is the use of tissue homogenates. The tissues within the three-dimensional systems of the invention can be cultured in the presence of a test agent and harvested to obtain tissue homogenate preparations for use in enzyme analysis. Preparation of tissue homogenates is well known in the art and involves the steps of tissue homogenization and subcellular fractionation to yield two main fractions routinely studied in drug metabolism: the post-mitochondrial supernatant and the endoplasmic reticulum (microsomal) fraction.

For preparation of the post-mitochondrial supernatant, the tissue homogenate can be centrifuged as 12,500×g for 15 minutes to pellet intact cells, cell debris, nuclei and mitochondria. The resultant supernatant (the post-mitochondrial supernatant) is carefully decanted and contains the microsomal plus soluble fractions of the cell. Microsomal tissue fractions can be prepared from the post-mitochondrial supernatant by one of two centrifugation techniques, one involving the use of an ultracentrifuge and the other involving a calcium precipitation of the microsomes at a lower g force.

The ultracentrifugation method uses aliquots (approximately 10-12 ml) of the post-mitochondrial supernatant, which are transferred to ultracentrifuge tubes and centrifuged at 100,000×g for 45 minutes in a refrigerated ultracentrifuge. After centrifugation, the supernatant is decanted and discarded and the microsomal pellet resuspended in a suitable buffer containing physiological concentrations of salt, such as Tris. This procedure yields the final microsomal suspension.

The calcium precipitation method is based on the calcium dependent aggregation of endoplasmic reticulum fragments and subsequent ‘low speed’ centrifugation of the aggregated microsomal particles. The advantages of this method are that it is less time-consuming and does not require an ultracentrifuge. Aliquots of post-mitochondrial supernatant are mixed with a final CaCl₂ concentration of 8 mM and left to stand on ice for 5 min, with occasional gentle swirling. The mixture is then centrifuged at 27,000×g for 15 min, the supernatant discarded and the pellet resuspended by homogenization in a buffer such as Tris at physiological pH, yielding the microsomal suspension.

The microsomal fractions prepared by both of the above methods may be further washed by resuspending the microsomal pellet in 0.1 M Tris buffer, pH 7.4, containing 0.15 M KCl to remove either adventitious protein or excess CaCl₂. The microsomal pellet can then precipitated as above and resuspended in Tris buffer. It is not mandatory to resuspend the final microsomal preparations in Tris buffer and other buffers such as phosphate may be used. When comparing tissue fractions for their ability to catalyze drug biotransformation, a measure of the tissue protein is advantageously made. Amongst several methods, protein is readily determined by the colorimetric method of Lowry et al. (1951), with reference to a standard curve of bovine serum albumin. The colored complex is a result of a complex between the alkaline copper-phenol reagent used and tyrosine and tryptophan residues of the protein, and can be detect by spectrophotometer at 705 nm. Other protein detection methods are well known in the art and include the Bradford assay.

Reduced nicotinamide adenine dinucleotide phosphate (NADPH) is often a necessary cofactor for many drug biotransformation reactions and serves as a source of reducing equivalents in the reaction (particularly hydroxylation and demethylation reactions).

As can be appreciated, a wide variety of cell types may be cultured in a flow system according to the invention including, but not limited to, cell types best grown in the presence of a secondary co-culture cell layer including, but not limited to, stem cells cultured in the presence of a cell feeder layer of fibroblasts; hepatocytes cultured in the presence of a layer of endothelial cells; or muscle cells cultured in the presence of a layer of endothelial cells. Other exemplary cell types include but are not limited to smooth or skeletal muscle cells, myocytes, cardiomyocytes, fibroblasts, chondrocytes, adipocytes, fibromyoblasts, ectodermal cells, including ductile and skin cells (kerotinocytes, dermal cells), proximal tubule epithelial cells, hepatocytes, kidney cells, pancreatic islet cells, cells present in the intestine, osteoblasts and other cells forming bone or cartilage, hematopoietic cells, and neuronal cells.

A radial flow system according the invention may also find application in the culture of stem cells. Stem cells have plasticity properties, that is stem cells from one tissue or organ can be induced to differentiate into cells of other organs, either in vitro or after transplantation in vivo. For example, stem cells from bone marrow, blood or placenta can differentiate into hepatic-like cells; this striking property of the stem cells provides new opportunities for overcoming the limitation of procuring human tissues such as liver for evaluating toxic potentials of novel therapeutics.

The quintessential stem cell is the embryonic stem (ES) cell, as it has unlimited self-renewal and pluripotent differentiation potential (Thomson, J. et al. 1995; Thomson, J. A. et al. 1998; Shamblott, M. et al. 1998; Williams, R. L. et al. 1988; Orkin, S. 1998; Reubinoff, B. E., et al. 2000). These cells are derived from the inner cell mass (ICM) of the pre-implantation blastocyst (Thomson, J. et al. 1995; Thomson, J. A. et al. 1998; Martin, G. R. 1981), or can be derived from the primordial germ cells from a post-implantation embryo (embryonal germ cells or EG cells). ES and/or EG cells have been derived from multiple species, including mouse, rat, rabbit, sheep, goat, pig and more recently from human and human and non-human primates (U.S. Pat. Nos. 5,843,780 and 6,200,806). When introduced into mouse blastocysts, ES cells can contribute to all tissues of the mouse (animal) (Orkin, S. 1998). Murine ES cells are therefore known to be pluripotent. When transplanted in post-natal animals, ES and EG cells generate teratomas, which again demonstrates their multipotency.

Embryonic stem cells are well known in the art. For example, U.S. Pat. Nos. 6,200,806 and 5,843,780 refer to primate, including human, embryonic stem cells that are stated to proliferate in an in vitro culture for over one year, maintain a karyotype in which the chromosomes are euploid and not altered through prolonged culture, maintain the potential to differentiate to derivatives of endoderm, mesoderm, and ectoderm tissues throughout the culture, and are inhibited from differentiation when cultured on a fibroblast feeder layer.

U.S. Patent Applications Nos. 20010024825 and 20030008392 describe human embryonic stem cells that are stated to proliferate in an in vitro culture for over one year, maintain a karyotype in which all the chromosomes characteristic of the human species are present and not altered through prolonged culture, maintain the potential to differentiate to derivatives of endoderm, mesoderm, and ectoderm tissues throughout the culture, and are inhibited from differentiation when cultured on a fibroblast feeder layer. U.S. Patent Application No. 20030113910 describes pluripotent non-embryonic stem cells, which are stated to be capable of proliferating in an in vitro culture for more than one year; maintain a karyotype in which the cells are euploid and are not altered through culture; maintain the potential to differentiate into cell types derived from the endoderm, mesoderm and ectoderm lineages throughout the culture, and are inhibited from differentiation when cultured on fibroblast feeder layers.

Stem cells of the present invention also include those known in the art that have been identified in organs or tissues (tissue specific stem cells). An adult stem cell is an undifferentiated cell found among differentiated cells in a tissue or organ, can renew itself, and can differentiate to yield the major specialized cell types of the tissue or organ. The primary roles of adult stem cells in a living organism are to maintain and repair the tissue in which they are found. Examples of adult stem cells for use with the culture systems of the present invention are described below. Hematopoietic stem cells give rise to all the types of blood cells: red blood cells, B lymphocytes, T lymphocytes, natural killer cells, neutrophils, basophils, eosinophils, monocytes, macrophages, and platelets. Bone marrow stromal cells (mesenchymal stem cells) give rise to a variety of cell types: bone cells (osteocytes), cartilage cells (chondrocytes), fat cells (adipocytes), and other kinds of connective tissue cells such as those in tendons. Neural stem cells in the brain give rise to its three major cell types: nerve cells (neurons) and two categories of non-neuronal cells—astrocytes and oligodendrocytes. Epithelial stem cells in the lining of the digestive tract occur in deep crypts and give rise to several cell types: absorptive cells, goblet cells, Paneth cells, and enteroendocrine cells. Skin stem cells occur in the basal layer of the epidermis and at the base of hair follicles. The epidermal stem cells give rise to keratinocytes, which migrate to the surface of the skin and form a protective layer. The follicular stem cells can give rise to both the hair follicle and to the epidermis. Stem cells of the present invention also include human induced pluripotent stem (iPS) cells.

Stem cells may require the use of a feeder layer of cells, typically fibroblasts. These fibroblasts release paracrine signals required to keep stem cells in their proliferative, undifferentiated state. In the present invention, the fibroblasts may be seeded on the flow side of the cell culture insert and stem cells seeded in the opposing, cell culture insert chamber. In this configuration, the effect of very precise cell signaling sequences on stem cell differentiation may be measured by introducing additives or signals (e.g., chemokines/cytokines) into the medium flow stream in a specific order. Stem cells may also require daily media changes to mitigate the spontaneous differentiating effect of metabolic waste accumulation.

In specific embodiments, culture systems of the invention can be used to conduct developmental toxicity risk assessments. One assay that can be employed is the EST assay (Embryonic Stem Cell Test), which discriminates amongst non, weak and strong embryotoxic compounds. The strategy is to find the drug concentrations which cause a 50% inhibition of differentiation into contracting cardiomyocytes and growth of embryonic stem cells (ID50 and IC50, respectively). A similar value for inhibition of growth (IC50) is determined for 3T3 cells. These three concentrations are inserted into three linear discriminant functions that define the embryotoxicity class; non, weak, and strong. In slightly adapted methods, assessment can be based on appropriate levels of tissue and developmental stage-specific molecular endpoint markers. These methods are well known in the art and are described, for example, by Genschow E, et al., ATLA 2004; 32:209-44; Newal D R, et al., Tox In Vitro 1996; 10:229-40; Scholz G, et al. Toxicol in Vitro 1999; 13:675-81; Scholz G, et al. Cells Tissues Organs 1999; 165:203-11, Bremer S, et al., Toxicology in Vitro 2001; 15:215-23; Bigot K, et al., Toxicology in Vitro 1999; 13:619-23; zur Nieden N I, et al., Toxicol in Vitro 2001; 15:455-61; and zur Nieden N I, et al., Toxicol Appl Pharmacol 2004; 194:257-69, the contents of which are incorporated herein by reference.

In another specific embodiment, cardiomyocytes derived from stem cells can be used with culture systems of the present invention for use in cardiotoxicology testing. Adult human heart tissue offers the most obvious stem cell source for generating cardiomyocytes. Beltrami and colleagues demonstrated in 2001 the existence of a slowly dividing population of cells, termed cardiac stem cells (CSC), in the normal, hypertrophied and post-infracted human heart (Beltrami A P, et al. Cell Biol Toxicol 2003; 114:763-76; Urbanek K, et al. Proc Natl Acad Sci USA 2003; 100:10440-5 and Urbanek K, et al. Proc Natl Acad Sci USA 2005; 102:8692-7). These cells most likely represent a population of cells responsible for normal homeostasis in the heart which are mobilized upon tissue damage. Later work isolated a rare, c-kit+, Sca-1+, MDR1+, Lin−, telomerase positive population of small cells from adult heart in a variety of species including human (Beltrami A P, et al., Cell Biol Toxicol 2003; 114:763-76; Bearzi C, et al., Proc Natl Acad Sci USA 2007; 104:14068-73 and Matsuura K, et al. J Biol Chem 2004; 279:11384-91). These cells maintained a stable phenotype over months of passage (self-renewing), generated the three main cell types found in the heart (cardiomyocytes, smooth muscle cells and endothelial cells) and recapitulated both the self-renewal and multipotency from single cells at low frequency (clonogenic). Bone marrow-derived stromal stem cells or mesenchymal stem cells (MSC), extracted from the iliac crest of healthy volunteers, are an even more readily available source of human adult stem cells. Cardiomyocyte formation of MSCs has been induced in vitro with 5-azacytidine, dexamethasone/insulin/ascorbic acid or co-culture with cardiomyocytes. More recently, white adipose stromal cells have also been shown to exhibit spontaneous cardiogenic potential. Several human gestational or fetal sources of stem cells for cardiomyocyte differentiation exist. Both CD133+ hematopoietic stem cells and mesenchymal stem cells derived from human umbilical vein have been shown to differentiate into cardiomyocyte-like cells as determined by expression of cardiac specific transcription factors and structural proteins. Similarly, placental-derived stem cells have been shown to differentiate into cardiomyocytes following co-culture with fetal cardiomyocytes or ascorbic acid treatment. Human embryonic stem cells (hESCs) are well known in the art to generate cardiomyocytes. Human stem cell-derived cardiomyocytes present channels and proteins of interest at physiological levels in their normal context, thus making it possible to conduct in vitro cardiotoxicity assays in culture systems of the present invention. Cardiotoxicology assays known in the art such as those used to detect responses to oxidative stress, resistance to apoptosis and protection from ischemia, can be conducted using stem cell-derived cardiomyocytes with minimal modifications. hESC-derived cardiomyocytes have been shown to express functional hERG/KCNE2 channels, and can be used in testing for channel inhibition. Manually isolated, beating hESC-derived cardiomyocytes have been applied to microelectrode arrays (MEA) (Kehat I, et al., Cir Res 2002; 91:659-61; and Reppel M, et al. J Electrocardiol 2005; 38:166-70). Spontaneously contracting stem cell derived cardiomyocytes can be maintained in culture for extended periods of time (e.g., up to 3 months) potentially allowing repeated, noninvasive analysis or multiple analyses progressing from noninvasive to invasive.

In yet another specific embodiment, hepatocytes derived from stem cells can be used with culture systems of the present invention for use in hepatotoxicity screening. Human ESCs are one source of hepatocytes for use in toxicity screening. Lavon et al. in 2004, showed that an enriched population of hepatocyte-like cells could be developed from hESC that were allowed to spontaneously differentiate and then treated with acidic fibroblast growth factor (aFGF) to create conditions similar to those found in the normal embryonic hepatic milieu (Lavon N, et al. Differentiation 2004; 72:230-8). Dexamethasone, sodium butyrate insulin, aFGF, basic fibroblast growth factor (bFGF), hepatocyte growth factor (HGF), activina A, and oncostatin M can also be used to induce hepatocyte differentiation. Lee et al. demonstrated that hUSC can be differentiated in vitro into a variety of cell types including, hepatocyte-like cells (Lee O K, et al. Blood 2004; 103:1669-75). Bone-marrow mesenchymal stem cells (BMSC) also have the capacity to differentiate into several cell types including a hepatocyte-like cell type (Shu S N, et al., World J Gastroenterol 2004; 10:2818-22; Lange C, et al., Transplant Proc 2005; 37:276-9; Shimomura T, et al. Hepatol Res 2007; 37:1068-79; Sato Y, et al., Blood 2005; 106:756-63; Aurich I, et al., Gut 2007; 56:405-15 and Oh S H, et al., Gastroenterology 2007; 132:1077-87). Talens-Visconti et al. showed that when compared to BMSCs, adipose derived MSCs have an equivalent ability to differentiate along the hepatic lineage (Talens-Visconti R, et al., World J Gastroenterol 2006; 12:5834-45). Exemplary assays that may be especially amenable to the use of stem cell derived hepatocyte-like cells include steatosis, cholestasis, phospholipidosis, mitochondrial toxicity, oxidative stress and drug metabolism-mediated toxicity (e.g., identification of reactive or toxic metabolites). All of these assays have employed in vitro cultures of primary hepatocytes or cell lines to evaluate conditions of hepatotoxicity with promising predictive success (Davila J C, et al. Ann Rev Pharmacol Toxicol 1998; 38:63-96; and Davila J C, et al., In Hepatotoxicity: From Genomics to in vitro and in vivo Models.: John Wiley & Sons, Ltd; (Saura Sahu Ed); 1-30, 2007).

In other embodiments, transwell devices according the invention find use when configured as planar flow systems. Exemplary uses of a reactor configured as a planar flow system will now be described. Using an inverted cell culture insert, endothelial cells may be seeded on the flow side of the insert in a static drop of culture. After a suitable seeding time (e.g., 1-6 hours), the insert is inverted to its original position and placed in the reactor. A second cellular population, for instance smooth muscle cells, may then be seeded in the opposing, reactor chamber of the cell culture insert and the bioreactor then filled with medium. Medium flow is initiated and the culture is maintained for the desired incubation time. In this example, endothelial cells are exposed to the planar flow conditions facilitated by the bioreactor, a circumstance similar to physiologic conditions. Similarly, the smooth muscle cells are protected from the planar flow of the reactor while still experiencing the benefits of a constantly renewed stream of nutrient-laden medium. In addition, the medium that diffuses through the membrane is conditioned by the normally excreted products of the endothelial cells via the above described phenomenon of paracrine signaling.

As can be appreciated, a reactor in planar flow configuration offers the unique opportunity to study several vascular biology applications where the interaction between endothelial cells and smooth muscle cells are critical. One specific application of this type of study is the evaluation of drug-induced vascular injury (“DIVI”). This particular drug screening strategy is currently limited to in vivo studies. DIVI evaluations are typically used as an early indication of drug toxicity and are used to screen broad families of chemicals for their suitability for further trials. However, these trials rely heavily on the use of animals, which incur great costs in manipulation and maintenance. The presently described reactors configured as planar flow systems provide the opportunity to accurately model DIVI without the use of animals.

Based upon the foregoing, the present invention encompasses an in vitro method of assaying drug-induced vascular injury (DIVI) of a chemical entity. Such a method includes steps of: (a) providing a well-based flow system having a cell culture insert, as described herein; (b) seeding endothelial cells on a membrane positioned at a flow side of the cell culture insert; (c) seeding smooth muscle cells on an opposite side of the membrane of the cell culture insert, the endothelial cells and smooth muscle cells opposing each other across the membrane of the cell culture insert; (d) positioning the cell culture insert in the well-based flow system such that a planar flow of culture medium is directed to the endothelial cells at the flow side of the cell culture insert; (e) including a chemical entity to be assayed for DIVI in the culture medium being directed by radial flow to the flow side of the cell culture insert; and (f) evaluating DIVI in the smooth muscle cells and endothelial cells of the cell culture insert relative to a control cell culture insert prepared by steps (a)-(d) but not exposed to said chemical entity. DIVI is indicated by increased pathology in the smooth muscle cells and endothelial cells of the cell culture insert exposed to the chemical entity relative to the control cell culture insert.

Although there have been shown and described particular embodiments of the invention, it will be obvious to those skilled in the art that various changes and modifications can be made therein without departing from the scope of the invention defined by the appended claims.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A well-based flow system for cell culture comprising:
an input;
a flow assembly in fluid communication with the input, the flow assembly configured to provide flowing medium to a cell culture insert; and
an output in fluid communication with the flow chamber to drain medium from the flow chamber.

2. The well-based flow system of claim 1, wherein the flow chamber is configured to generate a radial flow medium flow path.

3. The well-based flow system of claim 1, wherein the flow chamber is configured to generate a planar flow medium flow path.

4. A well plate for holding a plurality of cell culture inserts including a membrane, the well plate comprising:
a plurality of inlets positioned on an outside perimeter of the well plate;
a plurality of inlet conduits each connected to one of the plurality of inlets;
a plurality of wells each connected to one of the plurality of inlet conduits and each including a flow assembly and a flow chamber, the flow assembly configured to provide flowing medium to the flow chamber,
a plurality of outlet conduits each connected to one of the plurality of wells; and
a plurality of outlets on the outside perimeter of the well plate, each of the plurality of outlets connected to one of the plurality of outlet conduits.

5. The well plate of claim 4, wherein the plurality of wells are configured to generate a radial flow medium flow path in the flow chamber.

6. The well plate of claim 5, wherein the plurality of wells are configured to generate a planar flow medium flow path in the flow chamber.

7. A method of culturing cells, comprising:
(a) providing a well-based flow system according to claim 1 having a cell culture insert;
(b) seeding cells to be cultured on a membrane of said cell culture insert; and
(c) positioning said cell culture insert in the well-based flow system such that a flow of culture medium is directed to the flow side of the cell culture insert, said cells to be cultured maintained on said membrane of the cell culture insert.

8. The method according to claim 7, wherein the flow of culture medium directed to the flow side of the cell culture insert is a radial flow of culture medium.

9. The method according to claim 7, wherein the flow of culture medium directed to the flow side of the cell culture insert is a planar flow of culture medium.

10. A method of culturing primary cells in the presence of a secondary co-culture cell layer, comprising
(a) providing a well-based flow system according to claim 1 having a cell culture insert;
(b) seeding a secondary co-culture cell layer on a membrane positioned at a flow side of the cell culture insert;
(c) seeding primary cells to be cultured on an opposite side of the membrane of said cell culture insert, said secondary co-culture cell layer and primary cells to be cultured opposing each other across the membrane of the cell culture insert; and
(d) positioning said cell culture insert in the well-based flow system such that a flow of culture medium is directed to the flow side of the cell culture insert, said primary cells to be cultured maintained in the cell culture insert.

11. The method according to claim 10, wherein the flow of culture medium directed to the flow side of the cell culture insert is a radial flow of culture medium.

12. The method according to claim 10, wherein the flow of culture medium directed to the flow side of the cell culture insert is a planar flow of culture medium.

13. The method according to claim 10, wherein the secondary co-culture cell layer includes fibroblasts and the primary cells to be cultured comprise stem cells.

14. The method according to claim 10, wherein the secondary co-culture cell layer includes endothelial cells and the primary cells to be cultured comprise hepatocytes.

15. The method according to claim 10, wherein the secondary co-culture cell layer includes endothelial cells and the primary cells to be cultured comprise muscle cells.

16. An in vitro method of assaying liver toxicity of a chemical entity, comprising
(a) providing a well-based flow system according to claim 1 having a cell culture insert;
(b) seeding endothelial cells on a membrane positioned at a flow side of the cell culture insert;
(c) seeding hepatocytes on an opposite side of the membrane of said cell culture insert, said endothelial cells and hepatocytes opposing each other across the membrane of the cell culture insert;
(d) positioning said cell culture insert in the well-based flow system such that a radial flow of culture medium is directed to the endothelial cells at the flow side of the cell culture insert;
(e) including a chemical entity to be assayed for liver toxicity in the culture medium being directed by radial flow to the flow side of the cell culture insert; and
(f) quantitating death of hepatocytes on inside of the cell culture insert relative to a control cell culture insert prepared by steps (a)-(d) but not exposed to said chemical entity, wherein liver toxicity is indicated by increased death of hepatocytes in said cell culture insert exposed to the chemical entity relative to the control cell culture insert.

17. An in vitro method of assaying drug-induced vascular injury (DIVI) of a chemical entity, comprising
(a) providing a well-based flow system according to claim 1 having a cell culture insert;
(b) seeding endothelial cells on a membrane positioned at a flow side of the cell culture insert;
(c) seeding smooth muscle cells on an opposite side of the membrane of said cell culture insert, said endothelial cells and smooth muscle cells opposing each other across the membrane of the cell culture insert;
(d) positioning said cell culture insert in the well-based flow system such that a planar flow of culture medium is directed to the endothelial cells at the flow side of the cell culture insert;
(e) including a chemical entity to be assayed for DIVI in the culture medium being directed by planar flow to the flow side of the cell culture insert; and
(f) evaluating DIVI in the smooth muscle cells and endothelial cells of said cell culture insert relative to a control cell culture insert prepared by steps (a)-(d) but not exposed to said chemical entity, wherein DIVI is indicated by increased pathology in the smooth muscle cells and endothelial cells of said cell culture insert exposed to the chemical entity relative to the control cell culture insert.

18. A kit comprising the well-based flow system of claim 1 and instructions for use.

19. The kit of claim 18, wherein the instructions are in accordance with the method of claim 7.

20. The kit of claim 18, wherein the instructions are in accordance with the method of any one of claim 10.

21. The kit of claim 18, wherein the instructions are in accordance with the method of 16.

22. The method of claim 7, further comprising obtaining the well-based flow system.
